(19) United States
(12) Patent Application Publication
(10) Pub. No.: US 2010/0042229 A1
(43) Pub. Date: Feb. 18, 2010

(54) Activity specific finger prosthesis

(76) Inventor: Douglas C. Hawk, Parryville, PA (US)

Correspondence Address: RUTH EURE, 4795 EDISON AVENUE, BOULDER, CO, 80301, US

(21) Appl. No.: 12/459,530

(22) Filed: Jul. 3, 2009

Related U.S. Application Data
(60) Provisional application No. 61/134,009, filed on Jul. 3, 2008.

(51) Int. Cl. A61F 2/54

(52) U.S. Cl. 623/65

(57) ABSTRACT

Activity specific finger prostheses includes rubber thimble-like sockets that are sized and shaped to be positioned over an amputated finger residuum. Each socket has an activity specific tool attached to the distal end thereof. A display stand is included for storing the sockets and their respective tool during periods of non-use. Posts extend upward from the display stand top surface, wherein each socket has its own post. A formfitting insert is monolithically formed with at the socket distal tip. The base of each tool is fastened firmly to the insert. The tools include a jackknife blade, a bottle-opener/slot screwdriver, a saw blade, a heavy wire utility hook, a Phillips screwdriver, scissors, a can-opener and a corkscrew. A miniature rubber or plastic phallus or personal contoured massage device is also included, anchored to a rubber fingertip.

### CLAIM OF PRIORITY

The present application claims priority from U.S. Provisional Application Ser. No. 61/134,009, filed Jul. 3, 2008, of common inventorship herewith.

### FIELD OF THE INVENTION

This invention relates to finger prostheses and, more particularly, to activity specific finger prostheses for providing persons with one or more finger amputations with an effective means of performing their activities of daily living (ADLs).

### BACKGROUND OF THE INVENTION

The prior art has put forth several designs for prosthetic fingers as well as several designs for fingertip tools. A representative sampling of this art includes:

U.S. Pat. No. 5,147,386 to Roger Carignan describes a securable positioning finger prosthesis comprising a joint which is movable in a plurality of directions.

U.S. Pat. No. 4,332,178 to John Vulkich describes an extendible finger wrench comprising a wrench which is attachable to a finger.

U.S. Pat. No. 6,065,480 to Phillip J. Mader describes a digital prosthesis for dental flossing. This device comprises an attachment means to a finger and means for securing and feeding dental floss.

U.S. Pat. No. 5,522,903 to Constantin Sokolow et al describes a finger prosthesis which screws into the intramedullary canal of a phalange.

None of these references, alone or combined suggest or describe the present invention.

### SUMMARY OF THE INVENTION

It is an object of the present invention to provide an improved prosthetic device for attachment to the first or second phalange of a human finger.

It is a further object of the present invention to provide an improved prosthetic device for attachment to the first or second phalange of a human finger further comprising a useful tool such as a saw blade, scissors, knife phillips' head screwdriver, corkscrew, fishhook, can opener, flat head screwdriver, or phallus or personal contoured massage device, for example.

The human body involves a skeletal frame that supports the flesh, the composite of which involves bone, muscle, fat, and a covering of skin, and the total of which has a certain mass and firmness associated with rigidity. In other words, the parts and limbs of the human body have certain expected physical qualities as well as mechanical functions which enable man to perform various tasks throughout the course of a day. Some of the most versatile, and sometimes least appreciated appendages, are the fingers of the hand. Although small in relation to the size of the human body, one's fingers allows a person to interact with the environment around them by grasping, pulling, pushing and otherwise manipulating items/objects about them.

Typically, it is only with the loss of one or more fingers that an individual realizes the important role fingers play in their daily life.

Various attempts have been made in the prior art to replace amputated fingers with a prosthesis, much as one would an amputated leg or arm. However, it has been found, regardless of their simplistic appearance, that the motions performed by fingers are inherently difficult to imitate with prosthetic replacements. This is partly due to the many small joints, muscles and tendons that make up the anatomy of the human finger, and which can not adequately be replicated by a prosthesis.

Accordingly, a need remains for activity specific finger prostheses in order to overcome the above-noted shortcomings. The present invention satisfies such a need by providing an assembly that is convenient and easy to use, is durable yet lightweight in design, is versatile in its applications.

### BRIEF DESCRIPTION OF THE DRAWINGS

The novel features believed to be characteristic of this invention are set forth with particularity in the appended claims. The invention itself, however, both as to its organization and method of operation, together with further objects and advantages thereof, may best be understood by reference to the following description taken in connection with the accompanying drawings in which:

The figures generally include various perspective views showing activity specific finger prostheses, in accordance with the present invention.

FIG. 1 is a perspective view of the finger prostheses of the present invention mounted on a circular display stand.

FIG. 2 is a perspective view of a finger prosthesis being used to open a bottle.

FIG. 3 is a perspective view of the finger prostheses of the present invention spread out for display in juxtaposition to a human hand for which the prostheses are useful.

FIG. 4 is a perspective view of a finger prosthesis being used to saw wood.

FIG. 5 is a perspective view of a finger prosthesis being used to uncork a bottle of wine.

FIG. 6 is a perspective view of a finger prosthesis being used to hold a toy fish.

FIG. 7 shows a bottom view of the circular display stand.

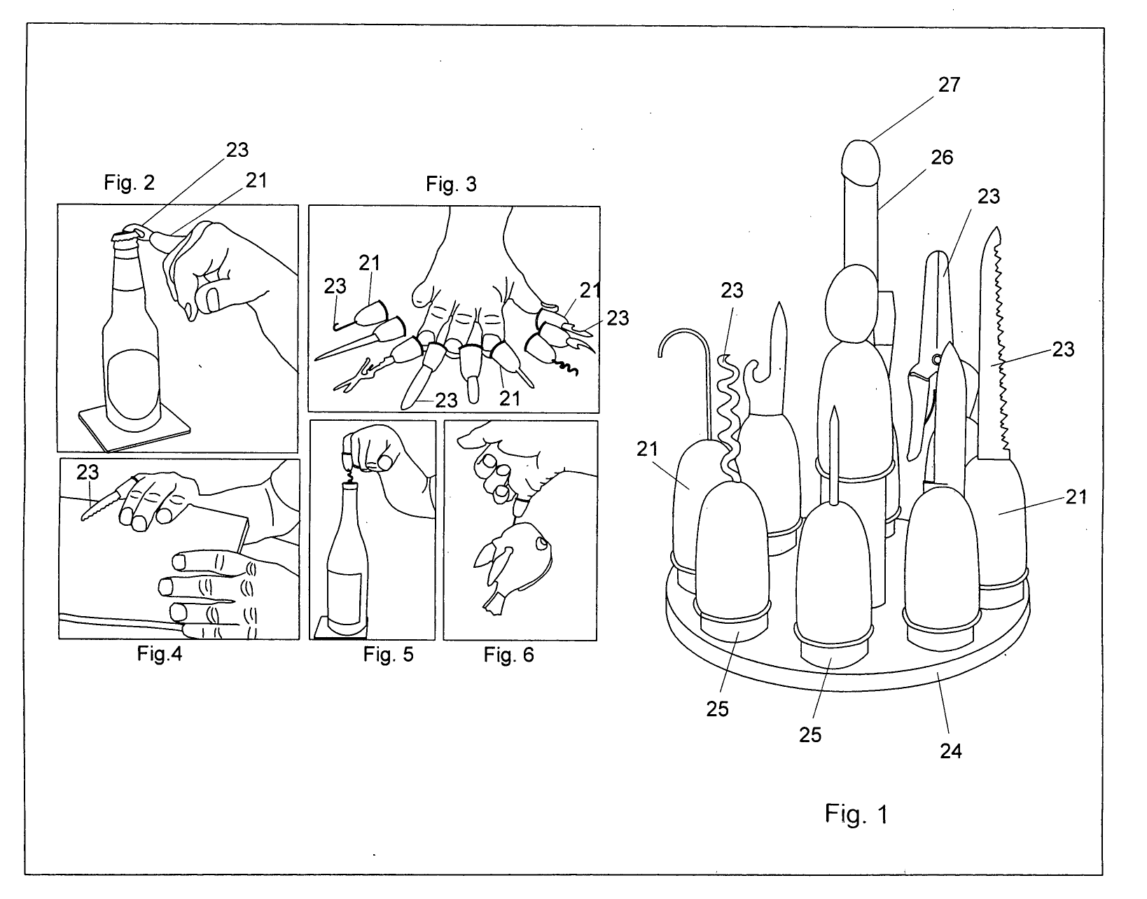

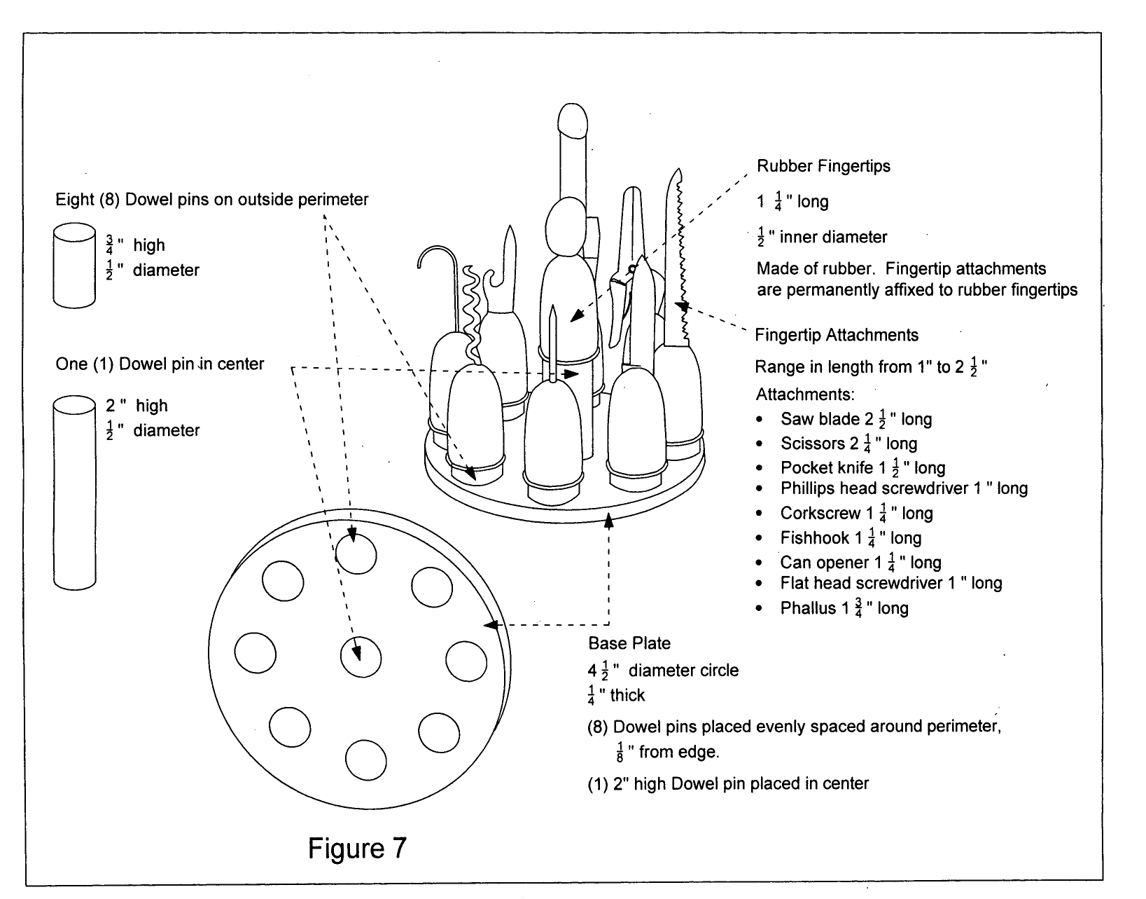

### DETAILED DESCRIPTION OF THE INVENTION

The present invention will now be described more fully hereinafter with reference to the accompanying drawings, in which a preferred embodiment of the invention is shown. This invention may, however, be embodied in many different forms and should not be construed as limited to the embodiment set forth herein. Rather, this embodiment is provided so that this application will be thorough and complete, and will fully convey the true scope of the invention to those skilled in the art.

The assembly 10 of this invention is referred to generally in the figures and is intended to provide activity specific finger prostheses 20. It should be understood that the assembly 10 may be used to provide prostheses for various activities and should not be limited to the activities set forth herein. Referring to the figures in general, the assembly 10 includes a plurality of rubber thimble-like sockets 21 that are suitably sized and shaped to be positioned over the residuum of an amputated finger 11. Such sockets 21 remain suspended from the residual appendage by suction created between the elastic socket 21 and the skin of the residual appendage. Each socket 21 has an activity specific tool 23 attached to the distal end thereof. A circular-shaped display stand 24 is included for storing the sockets 21 and their respective tool 23 during periods of non-use. A plurality of posts 25 extend upward from the display stands' 24 top surface wherein each socket has its own post. A formfitting insert 26 is monolithically formed with at the distal tip of each socket 21, wherein the base of each tool 23 is fastened firmly to the insert. Such tools 23 include a large jackknife utility blade, a bottle-opener/slot screwdriver, a saw blade, a heavy wire utility hook, a Phillips screwdriver, scissors, a can-opener and a corkscrew. Also included is a rubber or plastic phallus or personal contoured massage device 26, anchored, like the other items, to a rubber fingertip 27, which is important for creating a humorous scenario upon presenting an amputee with the assembly 10.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

It is noted the purpose of the foregoing abstract is to enable the U.S. Patent and Trademark Office and the public generally, especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

While the invention has been described with respect to certain specific embodiments, it will be appreciated that many modifications and changes may be made by those skilled in the art without departing from the spirit of the invention. It is intended, therefore, by the appended claims to cover all such modifications and changes as fall within the true spirit and scope of the invention.

In particular, with respect to the above description, it is to be realized that the optimum dimensional relationships for the parts of the present invention may include variations in size, materials, shape, form, function and manner of operation. The assembly and use of the present invention are deemed readily apparent and obvious to one skilled in the art.

1. Activity specific finger prostheses for providing persons with one or more finger amputations with an effective means of performing their activities of daily living.

2. A prosthetic device having an open proximal end and a distal end, wherein the distal end has a terminus, said device suitable for attachment to the first or second phalange of a human finger wherein the device is equipped with the head of a tool at the terminus of its distal end.

3. The prosthetic device of claim 2 wherein the tool is a screwdriver.

4. The prosthetic device of claim 2 wherein the tool is a saw blade.

5. The prosthetic device of claim 2 wherein the tool is a scissors.

6. The prosthetic device of claim 2 wherein the tool is a pocket knife.

7. The prosthetic device of claim 2 wherein the tool is a phillips' head screwdriver.

8. The prosthetic device of claim 2 wherein the tool is a corkscrew.

9. The prosthetic device of claim 2 wherein the tool is a fishhook.

10. The prosthetic device of claim 2 wherein the tool is a can opener

11. The prosthetic device of claim 2 wherein the tool is a personal contoured massage device.
